(19) United States
(12) Patent Application Publication
(10) Pub. No.: US 2008/0145439 A1
(43) Pub. Date: Jun. 19, 2008

(54) NANOPARTICLE DRUG FORMULATIONS

(75) Inventors: Thomas J. Lobl, Valencia, CA (US); John V. Schloss, Saugus, CA (US); Anna Imola Nagy, Valencia, CA (US); Jacob E. Pananen, Pasadena, CA (US)

Correspondence Address: BANNER & WITCOFF, LTD., 1100 13th STREET, N.W., SUITE 1200, WASHINGTON, DC, 20005-4051, US

(73) Assignee: NEUROSYSTEC CORPORATION, Valencia, CA (US)

(21) Appl. No.: 11/831,230

(22) Filed: Jul. 31, 2007

Related U.S. Application Data
(60) Provisional application No. 60/820,931, filed on Jul. 31, 2006.

(51) Int. Cl. A61K 9/51; A61K 31/4535; B01F 11/00

(52) U.S. Cl. 424/498; 424/501; 514/326; 366/108

(57) ABSTRACT

A suspension of nanoparticles in a liquid medium provides a mechanism for delivery of gacyclidine base or other drug that is substantially insoluble in the liquid medium.

### CROSS-REFERENCE TO RELATED APPLICATIONS

This application claims the benefit of U.S. Provisional Application Ser. No. 60/820,931, filed Jul. 31, 2006 and titled “Nanoparticle Drug Formulations and Delivery Thereof,” hereby incorporated by reference herein.

### BACKGROUND

It is well known that drugs work most efficiently in the body of a human or animal if they are delivered locally where needed. When delivered systemically there is a much greater chance for side effects, as all tissues are exposed to large quantities of the drug. Tissue-specific drug delivery can present challenges, however. In many cases, a target tissue (i.e., the tissue to be treated with a drug) is difficult to reach. Injecting a drug to such a target tissue may require a significant medical procedure which is both costly and unpleasant to the patient. If a drug treatment regimen requires delivery of small doses over a prolonged period, tissue-specific drug delivery may be impractical.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Forms of gacyclidine which are substantially insoluble can be delivered as a suspension of nanoparticles in a liquid medium. In some embodiments, nanoparticles are formed from a polymer or other material to which gacyclidine (and/or one or more other drugs) absorbs/adsorbs. In other embodiments, nanoparticles can be formed from gacyclidine (and/or one or more other drugs) in pure form, as a homogeneous mixture of gacyclidine (and/or one or more other drugs) and a polymer or other non-drug substance, or as a core of gacyclidine (and/or one or more other drugs) having a polymeric coating. Such nanoparticles and/or suspensions thereof can be formed in various manners, used for treatment of a variety of conditions, and delivered using various techniques.

### BRIEF DESCRIPTION OF THE DRAWINGS

The following detailed description of certain embodiments is better understood when read in conjunction with the accompanying drawings, which are included by way of example and not by way of limitation.

FIG. 1 shows a mixer according to at least some embodiments.

FIG. 2 is a cross-sectional view of the mixer of FIG. 1 with tubing omitted.

FIGS. 3 and 4 show a turbulence generator from the mixer of FIG. 1.

FIG. 5 shows an example of a drug delivery system.

FIG. 6 shows structure and solution chemistry of gacyclidine.

### DETAILED DESCRIPTION

### Targeted Delivery of Gacyclidine

Gacyclidine is an NMDA receptor antagonist useful in treating tinnitus. In addition to tinnitus therapy, gacyclidine has other potential therapeutic uses. NMDA receptor antagonists such as gacyclidine can prevent apoptosis of traumatized neurons and can protect neurons from intraoperative traumatic stress. Gacyclidine can therefore be used as an adjunct therapy for cochlear implant surgery, retinal implant surgery, neuromuscular implant surgery, or for other neurological surgery and/or in connection with other neurological implants. Numerous other uses for gacyclidine and formulations thereof are described in one or more of the commonly-owned U.S. patent applications having Ser. Nos. 11/337,815 (titled “Apparatus and Method for Delivering Therapeutic and/or Other Agents to the Inner Ear and to Other Tissues” and published as U.S. Pat. Pub. No. 2006/0264897), 11/759,387 (titled “Flow Induced Delivery from a Drug Mass” and filed on Jun. 7, 2007), 11/780,853 (titled “Devices, Systems and Methods for Ophthalmic Drug Delivery” and filed on Jul. 20, 2007), and 11/367,720 (titled “Improved Gacyclidine Formulations” and published as U.S. Pat. Pub. No. 2006/0205789).

Although gacyclidine has numerous therapeutic benefits when appropriately delivered, systemic delivery would expose all body tissues to effective doses, potentially resulting in undesirable side effects. It is therefore highly desirable to deliver gacyclidine directly to a cochlea, eye, brain region or other target tissue to be treated. To further reduce the potential for side effects, it may also be desirable to deliver gacyclidine in very small doses over a prolonged period (particularly when treating a chronic condition). As described in more detail below, prolonged small-dose delivery can be achieved with a device which is wholly or partially implanted within a patient. By implanting all or part of the drug delivery device, the patient is able to avoid repeated medical procedures.

Delivery of gacyclidine in such a manner presents numerous challenges, however. Various characteristics of gacyclidine are discussed in more detail below. Notably, however, many forms of gacyclidine that can be dissolved in a fluid medium for delivery to a target tissue are unstable. Other forms are more stable, but are highly insoluble and suffer loss (e.g., adsorption) to surfaces of catheters and other components. Specifically, catheters, drug reservoirs, pumps, anti-bacterial filters and other components of drug delivery systems are typically fabricated from polymers approved for human implantation. Gacyclidine will bind to many of these polymeric materials. Silicone, one of the most commonly used materials for human implantation, binds gacyclidine. At room temperature and in a gacyclidine solution at pH 6, silicone can retain up to 60% of 100 micromolar (μM) gacyclidine (depending on the time and temperature of exposure). Replacing all polymeric components of a drug delivery system with materials having little or no affinity for gacyclidine base can avoid drug loss to the delivery device, but this may not always be practical, and it will not necessarily resolve problems of drug stability.

In at least some embodiments, these challenges are addressed through the use of nanoparticles. As used herein (including the claims), “nanoparticle” refers to particles generally having a size of 200 nanometers (nm) or less, exclusive of temporary aggregation of such particles that might occur at high particle concentrations, Because of their size, Brownian motion will keep nanoparticles suspended in a fluid medium for a very long (or even indefinite) amount of time, and they can thus be used to carry forms of gacyclidine which are difficult to dissolve. Nanoparticles can also address problems caused by gacyclidine binding to device components. In particular, nanoparticles can be formed from materials that have a high affinity for gacyclidine and thus successfully compete with polymeric device surfaces for gacyclidine binding. For example, nanoparticles can tightly bind the stable basic form of gacyclidine and increase its thermal stability; a stable basic form of gacyclidine can be encapsulated such that less than 10% decomposition occurs in one year at 37° C. Nanoparticles can also maintain gacyclidine in a mobile phase that is capable of passing through an anti-bacterial filter that blocks particles larger than 0.22 μm. This can be an important element of a drug-delivery system in certain embodiments (e.g., when drug must be delivered to the cochlea or other tissue that is interconnected with the cerebrospinal fluid and central nervous system).

Using nanoparticles, it is possible to obtain a deliverable suspension having an effective concentration of an insoluble gacyclidine form that is much higher than would otherwise be possible. For example, a suspension having a 100 micromolar effective concentration of free gacyclidine base, a 1 millimolar (mM) effective concentration, a 100 millimolar effective concentration, or a higher effective concentration can be produced from a concentrated suspension of drug nanoparticles. As used herein (including the claims), “effective concentration” refers to a given volume of liquid containing as much of a particular compound (e.g., basic gacyclidine) in the suspended nanoparticles as that volume would contain if the same amount of the compound could be fully dissolved into that liquid. As also used herein (including the claims), “gacyclidine base” or “free base gacyclidine” includes all geometric isomers of free base gacyclidine, all optical isomers of free base gacyclidine, and all enantiomeric mixtures of free base gacyclidine.

In some embodiments, nanoparticles are formed from a polymer or other material to which gacyclidine (and/or another drug) absorbs/adsorbs. As used herein (including the claims) “absorbs/adsorbs” includes a nanoparticle absorbing another substance, a nanoparticle adsorbing another substance, and a nanoparticle both absorbing and adsorbing another substance. In other embodiments, nanoparticles can be formed from gacyclidine (and/or one or more other drugs) in pure form, as a homogeneous mixture of gacyclidine (and/or one or more other drugs) and a polymer or other non-drug substance, or as a core of gacyclidine (and/or one or more other drugs) having a polymeric coating.

### Nanoparticle Fabrication

Various methods can be employed to fabricate nanoparticles of suitable size. These methods include vaporization methods (e.g., free jet expansion, laser vaporization, spark erosion, electro explosion and chemical vapor deposition), physical methods involving mechanical attrition (e.g., the pearlmilling technology developed by Elan Nanosystems of Dublin, Ireland), and interfacial deposition following solvent displacement.

The solvent displacement method is relatively simple to implement on a laboratory or industrial scale and can produce nanoparticles able to pass through a 0.22 μm filter. The size of nanoparticles produced by this method is sensitive to the concentration of polymer in the organic solvent, to the rate of mixing, and to the surfactant employed in the process. Although use of the solvent displacement method with the surfactant sodium dodecyl sulfate (SDS) has yielded small nanoparticles (<100 nm), SDS is not ideal for a pharmaceutical formulation. However, similar natural surfactants (e.g., cholic acid or taurocholic acid salts) can be substituted for SDS to obtain similarly sized nanoparticles. Taurocholic acid, the conjugate formed from cholic acid and taurine, is a fully metabolizable sulfonic acid with very similar amphipathic solution chemistry to SDS. An analog of taurocholic acid, tauroursodeoxycholic acid (TUDCA), is not toxic and is actually known to have neuroprotective and anti-apoptotic properties. TUDCA is a naturally occurring bile acid and is a conjugate of taurine and ursodeoxycholic acid (UDCA). UDCA is an approved drug (ACTIGALL®, Watson Pharmaceuticals) for the treatment of gallbladder stone dissolution. Other naturally occurring anionic surfactants (e.g., galactocerebroside sulfate), neutral surfactants (e.g., lactosylceramide) or zwitterionic surfactants (e.g., sphingomyelin, phosphatidyl choline, palmitoyl carnitine) can be used in place of SDS or other surfactants that have been commonly employed in nanoparticle formulation studies. Other excipients that are generally recognized as safe, such as those used to solubilize the basic form of gacyclidine, can also be used to prepare nanoparticles. Such excipients include a polyoxyethylene fatty acid ester (e.g., polysorbate 80 (e.g., TWEEN 80®)), a polyglycol mono or di-ester of 12-hydroxy steric acid (e.g., SOLUTOL® HS 15), and CAPTISOL®. Poloxamers such as (but not limited to) poloxamer 407 can also be used.

A sampling of various surfactants can be used in order to determine the optimal surfactants for small (e.g., <100 nm), non-toxic drug-containing (e.g., gacyclidine) nanoparticles. Surfactant concentrations also affect the formation of the nanoparticles, their density and their size. A surfactant concentration can be optimized for each polymer composition, desired drug concentration, and intended use.

Of the various organic solvents previously employed in nanoparticle formulation, acetone is attractive because of its prior use in preparing filterable nanoparticles, its low toxicity, and its ease of handling. Various polymers composed of L- and D,L-lactic acid (PLA) or mixtures of lactic acid and glycolic acid (poly(lactide-co-glycolide)) (PLGA) are soluble in acetone, with the exception of 100% L-PLA and 100% glycolic acid (PGA). Polymers composed of 100% L-PLA will dissolve in methylene chloride and polymers composed of either 100% L-PLA or 100% PGA will dissolve in hexafluoroisopropanol (HFIP).

In some embodiments, rapid mixing is employed when preparing nanoparticles using the solvent displacement method. In some such embodiments, a stirring rate of 500 rpm or greater is typically employed. Slower solvent exchange rates during mixing result in larger particles. Fluctuating pressure gradients are used to produce high Reynolds numbers and efficient mixing in fully developed turbulence. Use of high gravity reactive mixing has produced small nanoparticles (10 nm) by achieving centrifugal particle acceleration similar to that achieved by turbulent mixing at high Reynolds numbers.

Sonication is one method that can provide turbulent mixing. Sonication is the method most commonly employed with the double emulsion nanoparticle fabrication method, but is less suited to the solvent displacement method. Sonication can be performed by mixing two liquid streams (e.g. one stream having dissolved particle polymeric material and the other stream having a drug and/or combination of drugs that will cause the particles to come out of solution and solidify) passing through a tube with an inline ultrasonic vibrating plate at the point of stream intersection. Formation of very small liquid droplets by vibrational atomization has also been employed in the fabrication of nanoparticles. For example, the DMP-2800 MEMS-based piezoelectric micropump (inkjet) system produced by the Spectra Printing Division (Lebanon, N.H.) of Dimatix, Inc. (Santa Clara, Calif.) forms a 10-50 pL (1-5×10⁻¹¹ liter) sized liquid droplet at 100,000 pL/s. Micropumps (inkjet systems) offer uniform mixing and the ability to reliably translate the process from lab to production scale, but production of nanoparticles smaller than 200 nm will still rely on mixing dynamics (i.e., the solidification timing of the precipitated solid or liquid intermediates produced on mixing) when piezoelectric micropumps are used to produce small, polymer-laden droplets. Temperature, surfactant and solvent composition are important variables in using this approach, as they modify the solidification dynamics and the density of the produced nanoparticle.

In additional embodiments, use of continuous flow mixers can provide the necessary turbulence to ensure small particle size. Various mixers have been described that can provide turbulent mixing on a sub-millisecond timescale. Such mixing devices include modified T-mixers such as the Berger mixer (described by R. L. Berger, B. Balko and H. F. Chapman in Rev. Sci. Instrum., 39:493-498 (1968)) or the Wiskind mixer (described by R. E. Hansen and M. W. Tonsager in J. Phys. Chem., 92:2189-2196 (1988)). The Wiskind mixer has a proven ability to achieve homogeneous mixing of two or more fluid streams during passage through the mixer. Use of such a system to prepare small nanoparticles (e.g., <100 nm) would allow for easy transition between laboratory scale and industrial scale production. Use of such mixing technology would also allow for the relatively simple development of a commercial process. Example 2 illustrates the preparation of nanoparticles from PLGA by rapid mixing.

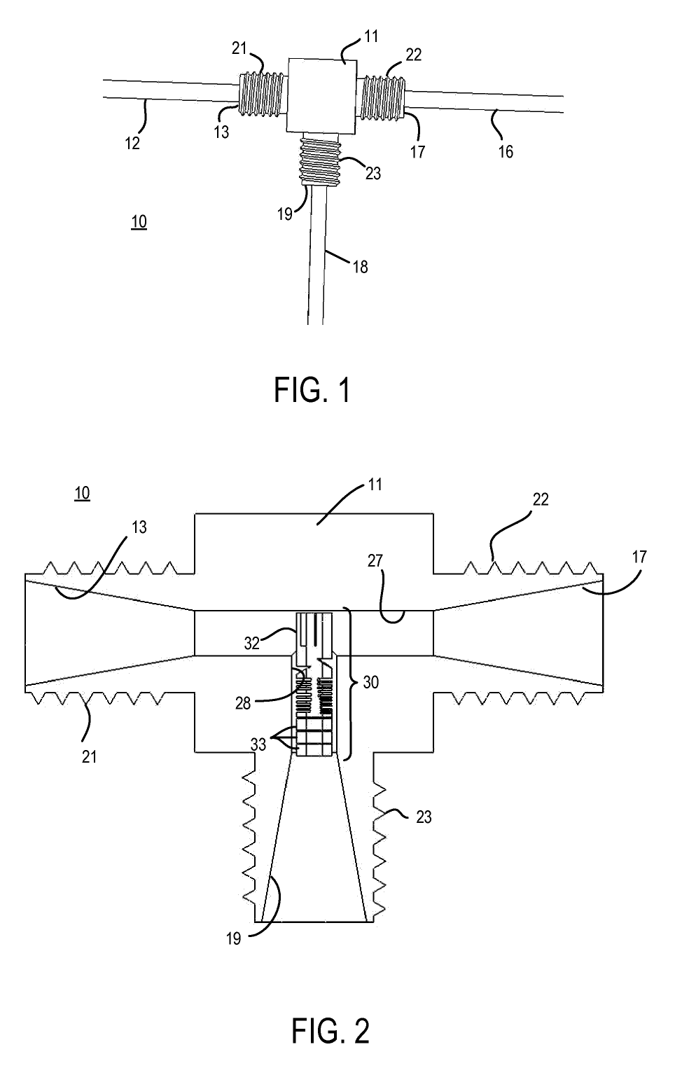

In some embodiments, a modified form of a Wiskind mixer is employed. FIG. 1 shows such a modified Wiskind mixer 10. Mixer 10 includes a PTFE (polytetrafluoroethylene) “Tee” connector 11 (e.g., part number K-06473-09 available from Cole-Palmer of Vernon Hills, Ill.). A first PTFE tubing line 12 connects to inlet 13, and a second PTFE tubing line 16 connects to inlet 17. An outlet PTFE tubing line 18 connects to outlet 19. Nuts and ferrules (not shown) are tightened around threads 21, 22 and 23 to secure lines 12, 16 and 18 in place and form fluid-tight connections. Tubing can be attached to inlets 13 and 17 and to outlet 19 so as to form fluid-tight connections in any of various other ways known in the art (e.g., use of o-ring compression fittings, adhesively bonding ends of tubes 12, 16 and 18 to inlets 13 and 17 and to outlet 19) In operation, fluids to be mixed are supplied through tubing 12 and 16 under pressure sufficient to cause fluid flow; the output in tubing 18 contains nanoparticles resulting from mixing (within connector 11) of the supplied fluids.

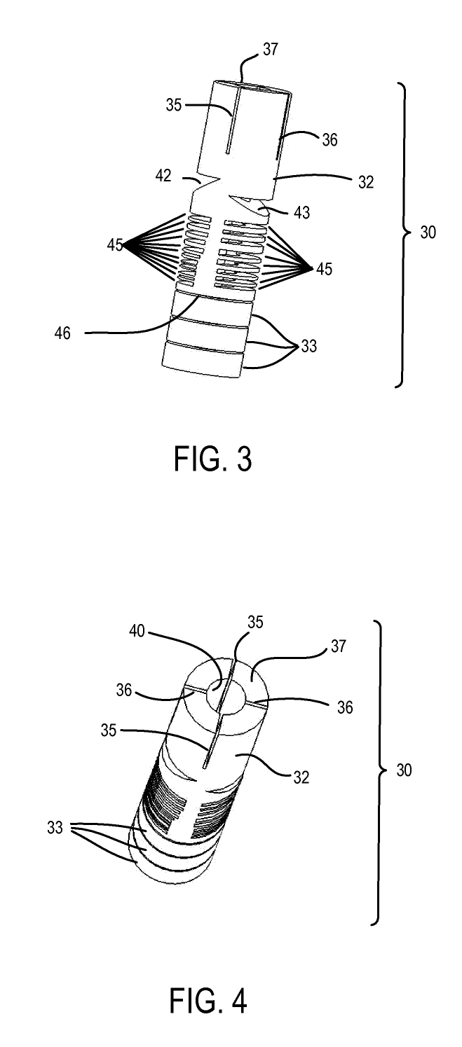

FIG. 2 is a cross-sectional view of mixer 10 in a plane parallel to that of FIG. 1. For convenience, tubing lines 12, 16 and 18 have been omitted in FIG. 2. A cylindrical bore 27 connects inlets 13 and 17, and is intersected by a second cylindrical bore 28 from outlet 19. Inlet 13, inlet 16 and outlet 19 are generally conical and can accept tubing with an outer diameter of between 1 and 4 mm. As also seen in FIG. 2, mixer 10 includes a turbulence generator 30. Turbulence generator 30 further includes a top portion 32 and three rings 33. FIG. 3 is an enlarged side perspective view of turbulence generator 30. FIG. 4 is a top perspective view of turbulence generator 30.

Turbulence generator 30 is in one embodiment formed from 22 gauge FEP (tetrafluoroethylene-hexafluoropropylene copolymer) tubing, and extends from the top of bore 27 to outlet 19. Cuts 35 and 36 in top portion 32 are 1 mm in length and provide access of the fluid streams from lines 12 and 16 to the interior 40 of turbulence generator 30. Angular openings 42 and 43 are located just below the access slits at 2 mm and 3 mm from top surface 37 of top portion 32. Multiple (10 to 15) cross-sectional slices 45 on opposite sides of top portion 32 are between 3 mm and 7 mm from top surface 37. Rings 33 are 1 mm in length and cut from the same type FEP tubing used to form top portion 32. Upon assembly of mixer 10, turbulence generator 30 is held in place by the end of tubing 18, which end is secured in outlet 19 by a nut around threads 23 (or by some other means). Rings 33 fill the space between the end of tubing 18 and the bottom edge 46 of top portion 32. A clearance exists between the inner surface of bore 28 and outer edges of top portion 32 and rings 33. A slight clearance also exists between top surface 37 and the upper inside of bore 27.

In at least one embodiment, the maximum volume available for liquid (after mixing the two inlet streams from lines 12 and 16) in connector 11 is less than 4 μL. Turbulence generator 30 creates a turbulent flow and efficient mixing after combining the two inlet streams. At a combined flow rate (i.e., the sum of the flow rates from lines 12 and 16) of 6 mL/min, the maximum time for fluid transit through the mixer is approximately 40 msec. At a combined flow rate of 60 mL/min, the maximum mixing time is approximately 4 msec. The actual time required to achieve homogeneous mixing is believed to be a small fraction of the maximum mixing time.

As previously indicated, there are at least four types of nanoparticles that can be used to deliver free base gacyclidine and/or other drugs: (1) nanoparticles formed from a polymer or other material to which gacyclidine (and/or another drug) absorbs/adsorbs or forms a drug coating on a polymeric nanoparticle core; (2) nanoparticles formed from gacyclidine and/or other drugs; (3) nanoparticles formed so as to comprise a generally homogeneous mixture of a drug with a polymer or other non-drug substance; and (4) nanoparticles of pure drug or drug mixtures with a polymeric coating over the therapeutic core. In some embodiments, a combination of rapid mixing (using mixer 10 of FIG. 1) and solvent displacement is employed to create nanoparticles of type (3). In one such embodiment, an erodible polymer (e.g., PLA, PGA, or PLGA) is dissolved in a water-miscible solvent (e.g., acetone). Free base gacyclidine is also dissolved in the water-miscible organic solvent. This solution of erodible polymer and gacyclidine dissolved in a water-miscible solvent is input through one of lines 12 or 16 and mixed with a suitable volume of water (input through the other of lines 12 and 16) to result in precipitation of the polymer as a particle. The water may have an added surfactant such as (but not restricted to) polysorbate 80. Gacyclidine base is trapped within the nanoparticles that form on mixing of the aqueous and organic solutions. The mixing time in some such embodiments is substantially less than 100 milliseconds, which will be less than the time taken for the mixed solutions to pass through the mixer. The shorter the time required to achieve homogeneous mixing, the smaller will be the diameter of resulting polymer particles.

In another embodiment, free base gacyclidine is dissolved in an organic solvent (e.g., ethanol) that will not dissolve purified nanoparticles composed of an erodible polymer. In this embodiment, gacyclidine base diffuses into the particles during incubation of the nanoparticles in the solution containing dissolved gacyclidine base. In yet another embodiment, nanoparticles of an erodible polymer are suspended in an aqueous solution of gacyclidine in the acid form or an aqueous solution/suspension of gacyclidine bound to a surfactant, e.g., polysorbate 80, which has a lower affinity for gacyclidine than the polymer. Over time, gacyclidine is absorbed/adsorbed to the suspended nanoparticles and subsequently diffuses into the particles.

Triglycerides, which have lower density than many polymers usable for nanoparticle formulation, can be included in the water-miscible organic solvent and/or subsequently imbibed by nanoparticles in suspension. Co-formulating triglycerides with polymers and gacyclidine can adjust the composite particle density to match the density of a vehicle (e.g., Ringer's solution, lactated Ringer's solution, physiological saline) used to deliver the nanoparticles to a target tissue. For example, addition of tryglycerides can yield nanoparticles having a density approximately equal to that of water (1 g/ml). Matching the densities of particles and vehicle will help to maintain the particles in a stable colloidal suspension for an extended period of time.

In another embodiment, type (1) nanoparticles (nanoparticles formed from a polymer or other material to which gacyclidine (and/or another drug) absorbs/adsorbs) of an erodible polymer (e.g., PLA, PGA, or PLGA) having a diameter less than 200 nm (or preferably, for certain embodiments, less than 100 nm) are suspended in a fluid (e.g., Ringer's solution or physiological saline, optionally containing a small amount of acid or surfactant). This suspension is then placed into contact with a fluidized bed of gacyclidine base microparticles (i.e., gacyclidine base particles larger than 1 micron in diameter). A portion of the gacyclidine base then absorbs/adsorbs into the nanoparticles, which nanoparticles can then be used to deliver the absorbed/adsorbed drug. If the nanoparticle suspension also contains acid or surfactant (e.g., polysorbate 80), such additives can act as mediators to facilitate transfer of gacyclidine from the solid particulate gacyclidine base to the polymeric nanoparticles.

In yet another embodiment, a particulate suspension of type (3) gacyclidine base nanoparticles (nanoparticles formed from pure gacyclidine, and/or gacyclidine combined with other drugs) is prepared by subjecting a mixture of solid free base of gacyclidine and a suitable vehicle (e.g., Ringer's solution or physiological saline) to high shear force. An example of such an embodiment is presented in Example 1. A surfactant is added to obtain a uniform dispersion of gacyclidine base in the vehicle. The size of the particles produced by this method depends on the shear force employed during high pressure homogenization, the concentration of gacyclidine base in suspension, the surfactant employed for dispersion, and the temperature. Particles of gacyclidine base smaller than 200 nm in diameter (which can be passed through an antibacterial filter as a suspension in a suitable vehicle) are formed. Particles of gacyclidine base larger than 200 nm in diameter, preferably larger than 1000 nm (1 μm) in diameter, can be used to prepare a fluidized bed, where such particles serve as an immobilized source of gacyclidine that can be eroded, eluted, or otherwise entrained in a mobile phase that flows past the particles.

In yet another embodiment nanoparticles of gacyclidine base can be prepared in the gas phase. Such gas phase preparation of nanoparticles composed of metals, metal oxides, or ceramics has been disclosed in U.S. Pat. Nos. 7,081,267, 7,052,777, and 6,855,426. These methods can be adapted for the preparation of nanoparticles of gacyclidine base by reducing the temperatures employed during nanoparticle fabrication. Dry nanoparticles of gacyclidine base can be stored as a sterile powder and reconstituted with a suitable vehicle (e.g., Ringer's solution or physiological saline).

In yet another embodiment nanoparticles of PLGA can be suspended in a solution of gacyclidine (or a mixture of gacyclidine and one or more other drugs) dissolved in a suitable solvent. This mixture of particles and drug are then disbursed as small droplets (e.g., through sieve) into a liquid nitrogen bath and quick frozen in liquid nitrogen. The frozen pellets are then lyophilized or evaporated, in vacuo, leaving behind the PLGA particles with gacyclidine (or a mixture of gacyclidine and one or more other drugs) on the surface. The particles are then re-suspended in a suitable vehicle in which gacyclidine (or a mixture of gacyclidine and one or more other drugs) is insoluble for delivery to the patient. In still another embodiment the PLGA particles can be atomized with the gacyclidine (or mixture of gacyclidine and one or more other drugs) and dried in vacuo as the droplets of drug and particles are suspended in the vacuum.

Type (1) nanoparticles of gacyclidine base can be coated with other substances, such as erodible polymers. Examples of erodible polymers include, but are not restricted to, polymers composed of PLA, PGA, or PLGA. Examples of methods for coating particles in the gas phase are disclosed in U.S. Pat. Nos. 7,052,777 and 6,855,426. Such methods can be adapted to the preparation of coated nanoparticles comprised of gacyclidine base, PLA, PGA, or PLGA, by reduction of the temperatures employed. Such particles can have several layers to provide sustained and controlled release of gacyclidine base from the drug-containing core over an extended period of time, e.g., over a period of weeks or months.

Polymers other than PLA, PGA and PLGA can be used in variations on the above embodiments (as well as in other embodiments). In some cases, a biodegradable polymer is preferred, though this is not necessary in some embodiments. Polymers with different rates of biodegradation (ranging from 2-3 weeks to 12-16 months) can be selected based on a particular application. Such polymers are available from Lakeshore Biomaterials of Birmingham, Ala. It is sometimes desirable that a chosen polymer have a higher affinity for the drug (e.g., gacyclidine) than other polymers the drug may encounter (e.g., in catheters, filters or containers), but this is also not always a requirement. In general, a selected polymer will usually be chemically stable as formulated at 37° C., non-toxic, and capable of forming uniform nanoparticles that will also form a stable suspension.

In addition to achieving isopycnic density with a vehicle (e.g., Ringer's solution), nanoparticles can be impregnated with triglycerides to increase their affinity for the basic form of gacyclidine. Such hybrid nanoparticles (e.g., PLGA-triglyceride) should be able to more effectively compete for adsorption of gacyclidine with polymeric surfaces such as silicone rubber components frequently used in implantable medical devices.

### Nanoparticle Sizing and Analytical Methods

Size-exclusion chromatography (SEC), also known as gel-filtration chromatography, can be used for analysis of gacyclidine-containing nanoparticles, both in the fractionation of such nanoparticles and/or in the pharmaceutical production of such nanoparticles. Various SEC media with molecular weight cut-offs (MWCO) for globular proteins ranging from 200,000 (Superdex 200) to about 1,000,000 (Superose 6) and to greater than 10⁸ (Sephacryl 1000) are suitable for SEC separation of viruses and small particles >1 μm and are commercially available (e.g., from GE Healthcare, Amersham Biosciences, Uppsala, Sweden).

In addition to SEC, layered semi-permeable membranes of progressively smaller pore sizes (e.g., tangential flow or CrossFlow membrane filtration) can be used to purify particles by size, to wash particles free of contaminating additives used in particle preparation, and/or to concentrate particles. Various companies such as, but not restricted to, Pall Corporation, Whatman, or Millipore, offer tangential flow, dead-end or tubular membrane systems that can be used to process particles for drug delivery. Such systems include nanofiltration, ultrafiltration (retention of 5,000 to 500,000 dalton-sized molecules), and microfiltration (retention of 0.2 to 0.45 μm diameter particles) membranes. Particles produced by the methodologies described herein can be freed of larger particles, unable to pass through an antibacterial filter, by passage through one or more microfiltration membranes and then concentrated to the desired concentration by use of an ultrafiltration or nanofiltration membrane. Use of membrane process technology can be done after adsorbing/absorbing drug to the particles, prior to impregnating particles with the desired amount of one or more drugs, or both.

The colloidal stability of nanoparticle suspensions may be assessed by accelerating sedimentation rates with a mini-centrifuge [Hermle Z229; 15,000 rpm; average g force 30,000×g (25,000-35,000×g)]. To a first approximation, a suspension that can retain colloidal dispersion for 20 minutes at 30,000×g should remain homogeneous for one year in an unstirred drug reservoir. Since PLGA, PGA and PLA have a density about 20% greater than water, nanoparticles fabricated from these materials should eventually sediment, rather than float to the surface. Co-formulating various oils of lighter density (e.g., olive or corn oil, triglycerides) with polymer and drug to match the density of the suspending liquid can also be performed. Such a formulation can maintain colloidal dispersion indefinitely.

### Example Devices and Methods for Drug Delivery Using Nanoparticles

In some embodiments, a needle, catheter end or other terminal component is surgically implanted into a target tissue of a patient and connected via a catheter (also implanted in the patient) to a subcutaneously implanted port. An implanted antibacterial filter may be included in the fluid path between the port and the terminal component. A preformulated suspension of drug-containing nanoparticles may then be injected into the port from an external source.

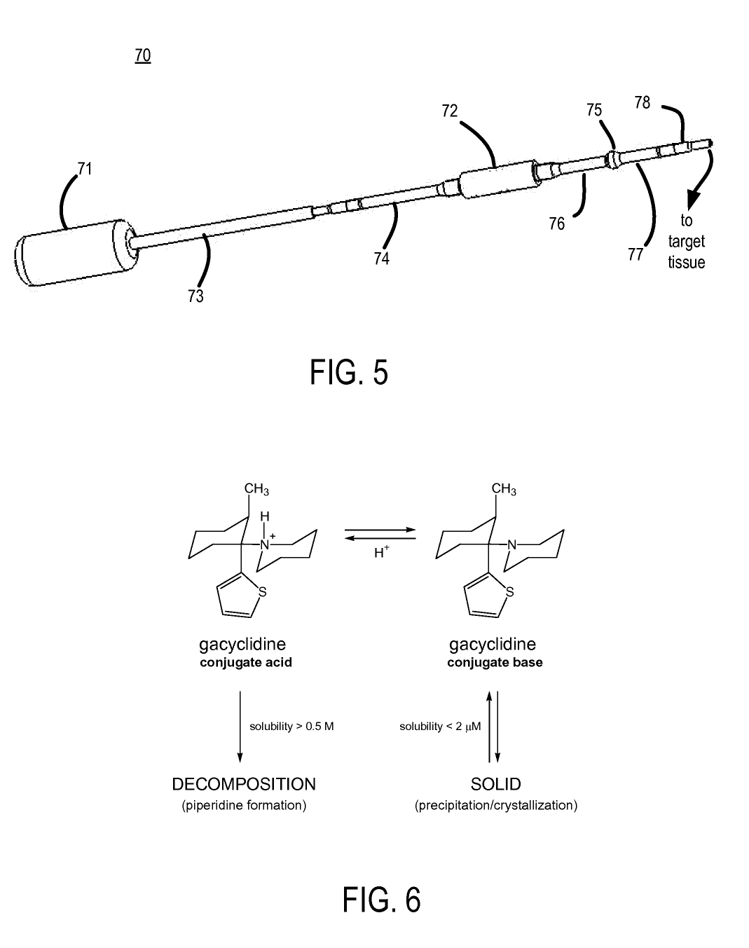

In other embodiments, a pump in fluid communication with a solid drug-containing reservoir is implanted in a patient and connected to a needle or other terminal component that has been implanted into a target tissue. A suspension of polymeric nanoparticles in an appropriate vehicle is passed over pellets of basic gacyclidine contained in the drug reservoir. The suspended nanoparticles absorb/adsorb gacyclidine from the reservoir and transport the absorbed/adsorbed drug to the target tissue. One such drug delivery system is shown in FIG. 5. In the embodiment of FIG. 5, system 70 includes an osmotic pump 71 coupled to a sleeved drug reservoir 72 via catheters 73 and 74. A three-dimensional (3-D) antibacterial filter 75 is coupled to drug reservoir 72 via a catheter 76. Another catheter 77 and connector 78 connects 3-D filter 75 via an additional catheter (not shown) to a terminal component (also not shown) positioned for delivery of a drug-containing nanoparticle suspension to a target tissue. The terminal component may be, e.g., a needle, an open end of a catheter, a cochlear implant, a retinal implant, etc.

Prior to implantation, osmotic pump 71 is filled with a suspension of polymeric nanoparticles having an affinity for gacyclidine in a suitable vehicle (e.g., Ringer's solution or physiological saline). Reservoir 72 is loaded with pellets of solid drug. The suspension is expelled from pump 71 and into reservoir 72 to absorb/adsorb gacyclidine from the gacyclidine pellets. The drug-laden nanoparticle suspension then passes through anti-bacterial filter 75 before reaching the terminal component and the target tissue. In another embodiment, smaller particles (e.g., 0.3 to 10 microns) of gacyclidine or other drug can be retained by an antibacterial filter to form a bed of drug substance. A suspension of nanoparticles passed through that filter would then simultaneously be sterile and absorb/adsorb drug. Still other embodiments employ a mixture of nanoparticles and dilute acid or amphipathic excipient flowing past solid drug particles. In still other embodiments nanoparticles composed of (or preloaded with) gacyclidine and/or other therapeutic agents can be used and the solid drug reservoir omitted.

Further embodiments include use of nanoparticles to deliver drugs using devices and/or methods described in the previously mentioned U.S. patent applications having Ser. Nos. 11/337,815, 11/759,387 and 11/780,853, as well as in commonly-owned U.S. patent application Ser. No. 11/414,543 (filed May 1, 2006 and titled “Apparatus and Method for Delivery of Therapeutic and Other Types of Agents”).

For applications where it may not be possible to adsorb sufficient quantities of gacyclidine during passage of nanoparticle suspensions through a drug chamber, a suspension of nanoparticles and gacyclidine base can be incubated for extended periods of time. Use of a solvent for gacyclidine that does not dissolve nanoparticles (e.g., ethanol) can be employed if dilute acid or inclusion of an amphipathic excipient fails to impregnate the nanoparticles with sufficient drug. In this approach, incubation of nanoparticles and gacyclidine can be conducted for extended periods of time.

Various embodiments further include nanoparticles comprising drugs in addition to (or instead of) gacyclidine, formation of nanoparticles of drugs in addition to (or instead of) gacyclidine and delivery of drugs in addition to (or instead of) gacyclidine using nanoparticles.

### ADDITIONAL EMBODIMENTS

In addition to the formulations, methods and devices described above, embodiments also include coating nanoparticles of pure drug with lipid to form unilamellar or multilamellar liposomes or vesicles, coating nanoparticles of pure drug with a membrane to control release of drug through the coating, and coating nanoparticles of pure drug with one or more erodable polymers which dissolve in vivo to release the drug.

As indicated above, certain embodiments of the invention include suspensions of nanoparticles. A suspension, as used herein (including the claims), includes mixtures of a liquid and nanoparticles in concentrations that effectively form a slurry. Various embodiments also include nanoparticle powders. In some such embodiments, less than 10% (by weight) of particles in the powder are larger than 200 nm. In still other embodiments, less than 1% (by weight) of particles in the powder are larger than 200 nm. In yet other embodiments, less than 10% (by weight) of particles in the powder are larger than 100 nm. In yet further embodiments, less than 1% (by weight) of particles in the powder are larger than 100 nm.

In certain embodiments, nanoparticles are used to deliver various drugs in combination with free base gacyclidine. Examples of such drugs that can be co-delivered with gacyclidine include, but are not limited to, NMDA antagonists other than gacyclidine (e.g., ketamine, caroverine, memantine, lidocaine, traxoprodil), subtype specific antagonists (e.g. NR2B and NR2D), steroids (e.g., dexamethasone, triamcinolone acetonide, methyl predinosolone), antiviral compounds (e.g., an antisense inhibitor, a ribozyme, fomivirsen, lamivudine, pleconaril, amantadine, rimantadine, an anti-idiotype antibody, a nucleoside analog), antibiotic compounds (e.g., an aminoglycoside, an ansamycin, a carbacephem, a carbapenem, a cephalosporin, a glycopeptide, a macrolide, a monobactam, a penicillin), antioxidants (e.g., N-acetyl-cysteine, glutathione, cysteine, methionine), and drugs identified in one or more of the above-mentioned commonly-owned U.S. patent applications. One or more of these drugs can be combined with gacyclidine to form nanoparticles of the combination (or of the combination and one or more polymers or other non-drug compounds). Nanoparticles formed from polymers (or other non-drug compounds) can also be used to elute gacyclidine and one or more of these additional drugs from pellets of gacyclidine and the additional drug(s). In still other variations, a nanoparticle formed from (or laden with) gacyclidine can be used to elute one or more other drugs from pellets of those one or more other drugs, or vice versa.

### Gacyclidine Characteristics

Gacyclidine (CAS Registry Numbers 131774-33-9 (acid form) and 68134-81-6 (base form)) has an apparent pK_{A} of 7.4 at room temperature in glass containers. FIG. 6 shows structure and solution chemistry of gacyclidine. However, in polymeric containers or in the presence of excess precipitated drug, the apparent pK_{A} is perturbed to lower values due to interaction of the basic form of the drug with polymeric surfaces or its own insoluble precipitate. In the conjugate acid form, gacyclidine is extremely water soluble. Solutions of greater than 1 molar (M) concentration can be prepared from the hydrochloride salt. However, solutions of the acid form of the drug are thermally unstable and subject to acid-catalyzed decomposition to give stoichiometric piperidine (1 mole per mole of drug lost) and multiple other organic products. The conjugate base form of gacyclidine is virtually insoluble in water (<2 μM above pH 9), but quite stable in aqueous suspension. Although the temperature dependence of gacyclidine's apparent pK_{A} is typical for an organic amine (−0.016 ΔpK_{A}/° C.), increased temperature dramatically increases the affinity of gacyclidine for polymeric surfaces and the rate of decomposition of its acid form.

The basic form of gacyclidine is 200-400 times more stable than the conjugate acid form (decomposition rates of 0.0013 day⁻¹ in 1 mM NaOH compared to 0.52 day⁻¹ in 10 mM HCl at 54° C.). The time required for 10% loss of gacyclidine in the hydrochloride salt form at body temperature (37° C.) is 3.8 days under slightly acidic conditions (see Table 1) and only slightly longer at physiological pH (6.1 days at pH 7.4; see Table 1). A completely implantable drug delivery system is achievable with the basic form of gacyclidine, where the anticipated time for 10% loss by decomposition should be greater than 2 years.

TABLE 1
Stability of Gacyclidine Hydrochloride Salt at 37° C.

| | Time for 10% Loss |
|---|---|
| pH | (37° C., Ringer's Lactate) |
| 5.5 | 3.7 ± 0.1 days |
| 6.0 | 3.82 ± 0.03 days |
| 7.4 | 6.1 ± 0.4 days |

The hydrochloride salt of gacyclidine gives a clear solution almost immediately on contact with water, up to a final concentration of >1 M. However, if a 0.1 M solution of the hydrochloride salt is diluted into a buffered aqueous solution to a final concentration of 1 mM, then precipitation of gacyclidine is observed above pH 7. By determining the residual gacyclidine in solution, an apparent pK_{A} of 6.7 is observed at room temperature. At room temperature and a final concentration of 1 mM gacyclidine, similar results are obtained in glass or polypropylene vials. However, at 37° C. the apparent pK_{A} observed in glass vials is about 0.7 pH units higher than the one observed in polypropylene, due to binding of gacyclidine base to the plastic vials. Inclusion of various amphipathic excipients (e.g., SOLUTOL HS 15, TWEEN 80 (polysorbate 80) or CAPTISOL) increases the amount of gacyclidine remaining in solution at high pH, without perturbing the apparent pK_{A}. Although various amphipathic excipients (e.g., SOLUTOL HS 15, TWEEN 80 or CAPTISOL) can increase the solubility of gacyclidine in glass containers, they are relatively ineffective in preventing loss of gacyclidine to polymeric surfaces, even at low pH.

### EXAMPLES

The following specific examples are provided for purposes of illustration only and are not intended to limit the scope of the invention.

### Example 1

### Preparation of a Suspension of Gacyclidine Base

The basic form of gacyclidine (200 mg) was suspended in 20 mL of Ringer's solution (Baxter Healthcare Corporation). Addition of 10 μL of polysorbate 80 to this suspension resulted in the achievement of a more uniform dispersion of gacyclidine base in Ringer's solution by use of a Dounce homogenizer. Two additional 10 μL aliquots of polysorbate 80 were added to the suspension to further improve the ability to obtain a uniform dispersion of gacyclidine base in Ringer's solution by use of the Dounce homogenizer. The sample was then subjected to treatment with a Labgen 700 Watt homogenizer for one cycle and a Microfluidics high pressure homogenizer for many cycles and then left to stand for 3 days. The volume-averaged particle size was 2847±715 nm. After standing for an additional 10 days at room temperature, the supernatant contained a low density of particles (≈1%). The supernatant was carefully removed from the pellet by aspiration and the settled large particle fractions were resuspended with 20 mL of Ringer's solution. The volume averaged size of the resuspended particles was 2808±621 nm and contained the gacyclidine. The large particle fraction contained 19 mM gacyclidine, which was completely retained by a 0.22 μm syringe filter (Millipore 0.22 μm PVDF Hydrophilic Syringe Filter Catalog No. SLGV0054SL).

### Example 2

### Use of a Modified Wiskind Mixer to Prepare PLGA Nanoparticles

Two different peristaltic pumps (L/S PTFE-tubing pump systems, Cole-Parmer Catalog number K-77912-00) were used to deliver aqueous solutions of surfactant and polymer dissolved in acetone to the two inlet positions of the Wiskind mixer described in connection with FIGS. 1-4. Aqueous solutions containing 0 to 10 g/L of polysorbate 80 were delivered at a flow rate of 6.3 mL/min by the first pump. Acetone solutions containing 0 to 20 g/L of 50:50 poly(lactide-co-glycolide) ester (PLGA, Lakeshore Biomaterials, 5050 DLG 3E, Lot No. LP271) were delivered at a flow rate of 1.0 mL/min by the second pump. The resultant particles formed during mixing of these solutions were assessed by determining the intensity of static and dynamic light scattering and the volume-averaged particle size (diameter) by use of a laser dynamic particle size analyzer (Horiba LB-550). The results of these mixing experiments are presented in Table 2. In the absence of polymer (PLGA) a low concentration of particles were formed by the surfactant alone. Polymer-derived particles increased in size at higher concentrations of polymer dissolved in acetone and decreased in size at higher concentrations of surfactant dissolved in water (Table 2). Even in the absence of surfactant, the volume-averaged diameter of polymer-derived particles was less than 100 nm (91±27 nm). When 10 g/L polysorbate 80 was included in the aqueous phase, the volume-averaged diameter of polymer-derived particles was 150 nm at a concentration of 20 g/L PLGA and 70 nm at a concentration of 2 g/L PLGA. Either size particles could pass through a 0.2 μm polyethersulfone (PES) antibacterial filter (VWR Catalog No. 28145-501) (Table 2). The smaller particles can be further purified by size exclusion chromatography (SEC) or microfiltration. Surfactant, if present during particle preparation, can be eliminated by ultrafiltration. After concentration to high density (e.g., 40% by weight) by ultrafiltration, these smaller particles can be impregnated with drug by suspension in a solvent that will dissolve drug (e.g., ethanol for gacyclidine), but will not dissolve suspended PLGA nanoparticles. The particles are soaked in the presence of dissolved drug(s) until the analysis shows the uptake of drug by the particles will have the desired drug(s) concentration/gm or volume of suspended particles. Following drug loading of the particles, the solvent and excess drug can be eliminated by further ultrafiltration or by use of tangential flow or CrossFlow membrane filtration.

TABLE 2
Use of Modified Wiskind Mixer to Produce Nanoparticles by
Continuous Flow Rapid Mixing

| Surfactant | Polymer | Stat | | |
|---|---|---|---|---|
| (g/L)¹ | (g/L)² | Scattering³ | Dyn Scattering³ | Size (nm)³ |
| 10 | 0 | 1.53 | 2.66 | 19 ± 4 |
| 10 | 20 | 1479 | 1989 | 150 ± 40 |
| 10 (filtered)⁴ | 20 (filtered)⁴ | 798 | 1137 | 150 ± 50 |
| 10 | 0 | 1.53 | 2.73 | 18 ± 4 |
| 10 | 2 | 46.69 | 71.82 | 66 ± 29 |
| 10 (filtered)⁴ | 2 (filtered)⁴ | 47.61 | 75.53 | 70 ± 28 |
| 5 | 0 | 1.53 | 2.37 | 15 ± 4 |
| 5 | 2 | 48.22 | 74.03 | 74 ± 26 |
| 2.5 | 0 | 0.92 | 1.73 | 13 ± 4 |
| 2.5 | 2 | 52.49 | 81.34 | 81 ± 28 |
| 1.25 | 0 | 0.61 | 1.47 | NPD³ |
| 1.25 | 2 | 54.93 | 71.49 | 82 ± 28 |
| 0.625 | 0 | 0.61 | 1.29 | NPD³ |
| 0.625 | 2 | 65.92 | 102.90 | 87 ± 28 |
| 0 | 0 | 0.61 | 1.29 | NPD³ |
| 0 | 2 | 45.78 | 71.09 | 91 ± 27 |

¹The concentration of polysorbate 80 dissolved in water.
²The concentration of 50:50 PLGA (5050 DLG 3E) dissolved in acetone.
³Stat Scattering = static scattering, scattered light intensity at the same wavelength as incident light; Dyn Scattering = dynamic scattering, scattered light intensity at a longer or shorter wavelength than incident light; Size = the volume-averaged particle diameter; NPD = no particles detected.
⁴After mixing, the nanoparticle suspension was filtered through a 0.2 μm PES filter.

### Example 3

### Formation of a Large Particle (>1 mm) of Gacyclidine Base and Delivery by Elution

Macroscopic solid pellets of drug base can be formed by melting the basic gacyclidine in a hot water bath (90-100° C.) then pipetting small amounts (2 μL) into a crystallization vial. By this method, uniform pellets of the basic form can be obtained (average weight 1.5±0.3 mg; average diameter 1.9 mm). These pellets can be loaded into a small flow chamber; then eluted with acid (HCl) at the desired drug concentration dissolved in the appropriate vehicle (e.g., Ringer's solution). A schematic for a prototype of such a device is illustrated in FIG. 5. Between the osmotic pump 71 (such as an ALZET® minipump (e.g., 2mL4, 2.5 μL/hr, 4 week duration for animal studies)) and an antibacterial filter 75 is a drug chamber 72 (2.1×8.5 mm; total volume 32 mm³) containing pellets of gacyclidine base (11 pellets; 18 mg gacyclidine base total). To prepare drug elution chambers suitable for use with the lower flow rate pumps (e.g., the DUROS® pump) smaller drug particles, such as those described in Example 1, can be used, such that the drug mass is sufficiently small. Alternatively, polymeric nanoparticles suspended in vehicle can be pumped from the osmotic pump to erode the drug from one or more solid free base pellets in chamber 72.

### Example 4

### Preparation of Concentrated Acetone-Free Nanoparticles

250 mL of 2 g/L PLGA dissolved in acetone was mixed at a flow rate of 1.0 mL/min with 10 g/L polysorbate 80 dissolved in water at a flow rate of 6.3 mL/min. After mixing, a Horiba LB-550 particle size analyzer was used to characterize the particles. The initial particle preparation had a static light scattering intensity of 45, a dynamic light scattering intensity of 71, and a volume-averaged particle diameter of 74 nm. This mixture was allowed to stand in an open beaker with constant stirring for 24 hours, after which there was no detectable odor of acetone. Particles were then concentrated to a final volume of 75 mL by use of pressure dialysis under nitrogen at 50 psi and a 10,000 MW cut-off cellulose membrane. As measured by the Horiba LB-550 particle size analyzer, the concentrated preparation had a static light scattering intensity of 472, a dynamic light scattering intensity of 750, and a volume-averaged particle diameter of 130 nm. The apparent increase in particle size after concentration was due to the reversible formation of particle oligomers (e.g., dimers and/or trimers) at higher particle concentration. Dilution of 0.2 mL of the concentrated particle preparation with 2.8 mL of water resulted in a measured decrease in the static light scattering intensity to 53, in the dynamic light scattering intensity to 89, and in the volume-averaged particle diameter to 53 nm.

### Example 5

### Loading of PLGA Nanoparticles with Gacyclidine Base

10 mL of 5 mg/mL microparticulate gacyclidine free base (volume-averaged particle diameter=2800 nm; 50 mg gacyclidine base), as described in Example 1, was added to 75 mL of the concentrated PLGA nanoparticle preparation described in Example 4 containing 0.5 g of PLGA and 0.75 g of polysorbate 80. The particles of gacyclidine base dissolved in the PLGA-polysorbate 80 suspension upon mixing. After addition of the microparticulate gacyclidine base, the mixture had a static light scattering intensity of 446, a dynamic light scattering intensity of 731, and a volume-averaged particle diameter of 130 nm. Incubation of this mixture at room temperature results in partitioning of gacyclidine between the dissolved polysorbate 80 and the colloidal PLGA nanoparticles. As a function of incubation time, gacyclidine absorbs/adsorbs to the PLGA nanoparticles and then diffuses into the particles. The distribution of gacyclidine between polysorbate 80 and PLGA nanoparticles can be determined by comparing the total gacyclidine content of the incubation mixture with the concentration of gacyclidine that will pass through a 10,000 molecular weight (MW) cut-off cellulose membrane, when the sample is subjected to pressure dialysis as described in Example 4. The concentration of gacyclidine in these solutions can be measured by a combination of methylene chloride extraction and HPLC.

### CONCLUSION

All patents and patent applications cited in the above specification are expressly incorporated by reference. However, in the event that one of said incorporated patents or applications uses a term in a manner that is different from the manner in which such term is used in the above specification, only the usage in the above specification should be considered (to the extent any language outside the claims need be considered) when construing the claims.

Numerous characteristics, advantages and embodiments of the invention have been described in detail in the foregoing description with reference to the accompanying drawings. However, the above description and drawings are illustrative only. The invention is not limited to the illustrated embodiments, and all embodiments of the invention need not necessarily achieve all of the advantages or purposes, or possess all characteristics, identified herein. Various changes and modifications may be effected by one skilled in the art without departing from the scope or spirit of the invention. Although example materials and dimensions have been provided, the invention is not limited to such materials or dimensions unless specifically required by the language of a claim. The elements and uses of the above-described embodiments can be rearranged and combined in manners other than specifically described above, with any and all permutations within the scope of the invention. As used herein (including the claims), “in fluid communication” means that fluid can flow from one component to another; such flow may be by way of one or more intermediate (and not specifically mentioned) other components; and such flow may or may not be selectively interruptible (e.g., with a valve). As also used herein (including the claims), “coupled” includes two components that are attached (movably or fixedly) by one or more intermediate components.

1. A composition comprising a suspension of nanoparticles in a liquid medium, wherein the suspended nanoparticles contain free base gacyclidine in an amount sufficient to achieve an effective concentration of free base gacyclidine in the suspension of at least 100 micromolar.

2. The composition of claim 1, wherein the effective concentration is less than 100 millimolar.

3. The composition of claim 1, wherein the nanoparticles consist of free base gacyclidine.

4. The composition of claim 1, wherein the nanoparticles include gacyclidine in a substantially homogeneous mixture with at least one polymer.

5. The composition of claim 4, wherein the at least one polymer includes at least one of L- or D,L-lactic acid (PLA), a mixture of lactic acid and glycolic acid (poly(lactide-co-glycolide)) (PLGA), and glycolic acid (PGA).

6. The composition of claim 1, wherein the nanoparticles are formed from at least one polymer having an affinity for free base gacyclidine, and wherein the free base gacyclidine contained in the nanoparticles is absorbed/adsorbed to the at least one polymer.

7. The composition of claim 6, wherein the at least one polymer includes at least one of L- or D,L-lactic acid (PLA), a mixture of lactic acid and glycolic acid (poly(lactide-co-glycolide)) (PLGA), and glycolic acid (PGA).

8. The composition of claim 1, wherein the nanoparticles further comprise a second drug.

9. The composition of claim 8, wherein the second drug is selected from the group comprising an NMDA antagonist other than gacyclidine, a subtype specific antagonist, a steroid, an antiviral compound, an antibiotic compound, and an antioxidant.

10. The composition of claim 8, wherein the second drug is selected from the group comprising ketamine, caroverine, memantine, lidocaine and traxoprodil.

11. The composition of claim 8, wherein the second drug is selected from the group comprising dexamethasone, triamcinolone acetonide and methyl prednisolone.

12. The composition of claim 8, wherein the second drug is selected from the group comprising an anti-viral compound or an antibiotic compound.

13. The composition of claim 12 where second drug is at least one antiviral compound selected from the group comprising lamivudine, pleconaril, amantadine, rimantadine, and a nucleoside analog.

14. The composition of claim 12 where the second drug is at least one antibiotic compound selected from the group comprising an aminoglycoside, an ansamycin, a carbacephem, a carbapenem, a cephalosporin, a macrolide, a monobactam, and a penicillin.

15. The composition of claim 8, wherein the second drug is selected from the group comprising N-acetyl-cysteine, glutathione, cysteine, and methionine.

16. The composition of claim 1, wherein the nanoparticles comprise a triglyceride in an amount sufficient to cause densities of the nanoparticles to be approximately equal to the density of the liquid medium.

17. The composition of claim 1, wherein the nanoparticles comprise a biodegradable polymer.

18. The composition of claim 17, wherein the biodegradable polymer comprises a first polymer having a first rate of biodegradation and a second polymer having a second rate of biodegradation, and wherein the first rate of biodegradation is different from the second rate of biodegradation.

19. The composition of claim 1, wherein the nanoparticles are coated with a lipid.

20. The composition of claim 1, wherein the liquid medium comprises Ringer's solution, lactated Ringer's solution, or physiological saline.

21. A method comprising:
directly delivering the composition of claim 1 to an inner ear of a human or animal.

22. A method comprising:
directly delivering the composition of claim 1 to an ocular tissue of a human or animal.

23. A method comprising:
directly delivering the composition of claim 1 to a neural tissue of a human or animal.

24. A mixer comprising:
a housing having first and second inlets and an outlet, the housing having one or more passages formed therein placing the first and second inlets and the outlet in fluid communication with one another; and
a turbulence generator positioned in the one or more passages, the turbulence generator including a main member having a bore formed therein and an upper end, wherein the turbulence generator includes at least one fluid entrance formed in the upper end and a plurality of apertures formed along a length of the main member.

25. The mixer of claim 24, wherein
the at least one fluid entrance formed in the upper end of the turbulence generator includes multiple cuts into an upper surface of the turbulence generator that are generally parallel to a longitudinal axis of the main member,
the plurality of apertures formed along the length of the main member include at least one angled cut that is non-orthogonal to the longitudinal axis of the main member and at least one cut generally orthogonal to the longitudinal axis of the main member, and
the turbulence generator further include a plurality of annular members positioned below and movable independent of the main member.

26. A method, comprising:
supplying, to the first inlet of the mixer of claim 24, a solution that includes an erodible polymer and a water-miscible solvent;
supplying water to the second inlet of the mixer of claim 24; and
recovering nanoparticles that include the erodible polymer from the outlet of the mixer of claim 24.

27. The method of claim 26, wherein the erodible polymer includes at least one of L- or D,L-lactic acid (PLA), a mixture of lactic acid and glycolic acid (poly(lactide-co-glycolide)) (PLGA), and glycolic acid (PGA).

28. The method of claim 26, wherein the solution that includes an erodible polymer and a water-miscible solvent further includes gacyclidine.

29. The method of claim 28, wherein the water supplied under pressure to the second inlet includes at least one surfactant selected from cholic acid or a salt thereof, ursodeoxycholic acid or a salt thereof, tauroursodeoxycholic acid or a salt thereof, taurocholic acid or a salt thereof, a poloxamer, polyvinyl alcohol, albumin, a polyoxyethylene fatty acid ester, a polyglycol mono or di-ester of 12-hydroxy steric acid, or a cyclodextran.

30. A method, comprising:
supplying under pressure, to the first inlet of the mixer of claim 24, a solution that includes gacyclidine and a water-miscible solvent;
supplying water under pressure to the second inlet of the mixer of claim 24; and
recovering nanoparticles that include gacyclidine from the outlet of the mixer of claim 24.

31. A composition comprising a nanoparticle powder, wherein the nanoparticles of the powder include free base gacyclidine, and wherein less than 10% (by weight) of all particles in the powder are larger than 200 nm.

32. The composition of claim 31, wherein the nanoparticles of the powder consist of free base gacyclidine.

33. The composition of claim 31, wherein the nanoparticles of the powder include gacyclidine in a substantially homogeneous mixture with at least one polymer.

34. The composition of claim 33, wherein the at least one polymer includes at least one of L- or D,L-lactic acid (PLA), a mixture of lactic acid and glycolic acid (poly(lactide-co-glycolide)) (PLGA), and glycolic acid (PGA).

35. The composition of claim 31, wherein the nanoparticles of the powder are formed from at least one polymer having an affinity for free base gacyclidine, and wherein the free base gacyclidine contained in the nanoparticles is absorbed/adsorbed to the at least one polymer.

36. The composition of claim 37, wherein the at least one polymer includes at least one of L- or D,L-lactic acid (PLA), a mixture of lactic acid and glycolic acid (poly(lactide-co-glycolide)) (PLGA), and glycolic acid (PGA).

37. The composition of claim 31, wherein the nanoparticles of the powder further comprise a second drug.

38. The composition of claim 37, wherein the second drug is selected from the group comprising an NMDA antagonist other than gacyclidine, a subtype specific antagonist, a steroid, an antiviral compound, an antibiotic compound, and an antioxidant.
